(19) United States
(12) Patent Application Publication
(10) Pub. No.: US 2021/0108164 A1
(43) Pub. Date: Apr. 15, 2021

(54) PUNCH DOWN APPARATUS

(71) Applicant: Gonzalo BALBAS PENAS, Burgos (ES)

(72) Inventor: Gonzalo BALBAS PENAS, Burgos (ES)

(21) Appl. No.: 17/063,882

(22) Filed: Oct. 6, 2020

(30) Foreign Application Priority Data
Oct. 15, 2019 (ES) ES201931685U

(51) Int. Cl. C12G 1/02; B01F 7/16; B01F 7/00; B01F 5/10

(52) U.S. Cl. CPC C12G 1/02; B01F 7/161; B01F 7/1625; B01F 2215/007; B01F 5/104; C12G 2200/25; B01F 7/00716

(57) ABSTRACT

The present invention relates to a punch down apparatus and rotating sprayer that is axially movable along its shaft through the supporting vat, also provided with an element for breaking the solid upper cover referred to as “cap” in the art.

### OBJECT OF THE INVENTION

The present invention relates to an all-in-one punch down apparatus and automatic rotating sprayer for the fermentation of fruits in vats.

The invention is primarily comprised in the field of application of installations for obtaining red wines of high quality and expression.

The present invention relates to a punch down apparatus and rotating sprayer that is axially movable along its shaft through the supporting vat, also provided with an element for breaking the solid upper cover referred to as “cap” in the art.

The invention relates to an element serving as an accessory for vats where the vinification or fermentation of fruits (generally grapes) takes place, in obtaining musts and for the improvement thereof.

It has a dual function, that is, the function of pumping over the must, spraying the cap, and the function of an element for breaking same, fragmenting and submerging the cap in the must and mixing it all with said must.

The object of the invention is to provide an apparatus and method to prevent the formation of the cap, as has been known up until now, and in turn optimize the exchange of the contents thereof, by continuous contact with the must, by pumping over same, and thus favoring product quality.

Given the applied technology, the arrangement, the applied ingenuity, and the design, the invention represents a breakthrough with respect to the current state of the art, particularly because of the applied concepts. Furthermore, the device is a high-performance and low-maintenance device.

### BACKGROUND OF THE INVENTION

It is known in the state of the art, and in particular in the production of wine as its main application, that in the fermentation of fruit, once it is broken up inside a vat, with all the parts such as the skins, must, pulp, stems, and seeds, basically each part with its own characteristics and different densities, in contact with one another, carbon dioxide is produced during fermentation. The less dense parts are pushed upwards and float, and as they accumulate and dry, they form a compact solid on the flotation line, referred to as the “cap.” This cap has a dense and compact structure, acting as a cover for the fermentation, CO2, aromas, and the heat that is accumulated, and it furthermore blocks the extraction and mixture of the compounds that are to release the active ingredients, such as tannins, anthocyanins, aromatic substances, and others, which are highly valuable for the mixture and are contained in the “cap” itself.

Once this “cap” has been dispersed or dissolved, it is characteristically formed over and over again very rapidly, every time it is dispersed, so it represents a considerable drawback as it limits the output or the releasing of the necessary compounds with respect to the must.

To somewhat prevent this from happening, in the past the cap was broken up with sticks or punch down apparatus, and later on “pumping over” was discovered. Pumping over consists of extracting the liquid part from the bottom of the vat, the must, and pouring it over the top part of the vat, on the “cap”, and therefore, through this recirculation, better mixing of the compounds is obtained and a larger amount of active ingredients are therefore transferred to the must.

Punch down apparatus: A generally wooden stick used to move the lees around when wine is fermented (punching down). Also stirrer. Element for performing punching down.

Punching down: Operation performed during alcoholic fermentation for the purpose of mixing solid and liquid particles, consisting of breaking up and stirring the cap during fermentation.

Both in the case of breaking up the cap and in the case of pumping over the must, a number of attempts, systems, and methods have been devised, such as cylinders, blades, pistons, screws, fluidized beds, etc., all in reference to many other patents and registrations.

The following, most representative registrations, among others, refer to the mentioned systems:

FR 2778411, ES 2379891, ES 2190301, ES 1063660, ES 2297144, U.S. Pat. No. 3,871,272 A, FR 2797783, ES 2181035, FR A 2767532, ES 0110914, and others using CO2 as a fluidized bed.

In no case are there any known systems that are as easily regulated as the invention.

### DESCRIPTION OF THE INVENTION

The proposed invention solves the drawbacks set forth in a very satisfactory manner, based on a simple, highly effective solution that makes use of the existing installations of vats and pumping over units.

The invention consists of a punch down apparatus-automatic rotating sprayer which moves up and down in the vat, mobilized by the pumping over of the must from the bottom of the vat.

An apparatus and method solving the drawbacks set forth in a simple and effective manner, which allows the mixing of the active ingredients to be improved, when desired, has been conceived. For that purpose, combined in a single element, the invention offers a dual function, that is, the function of pumping over the must, spraying the cap, and mixing the must, and the function of an element for breaking same, fragmenting and submerging the cap in the must, and in its cycle fragmenting the cap and diluting it in the must.

The proposed invention improves, optimizes, and solves the aforementioned deficiencies and drawbacks, as well as others, in a simple but effective and very satisfactory manner. For the different embodiments, the improvements are:

The cap is broken up by means of a generally pointed punch down apparatus, with a lower mass; the apparatus starts by breaking up the cap, being accompanied by radial spray arms, in the attempt to penetrate the must with same, all by introducing the assembly into the must.

The cap is sprayed, by way of a sprinkler, by pumping over the must, and during spraying, the fluid exiting the radii in a tangential manner towards the cap generates an automatic rotation, sweeping across the entire surface of the cap and of the vat.

While the radii break the cap up with their tangential outlet force of the must, they attempt to rotate, move, or break same.

When immersed in the fluid, the radii continue with their rotation force and stir the mixture of viscous and solid fluids.

In order to, finally, in the outlet thereof, break the cap up again on the other side with the sprayer radii, randomly, either continuing or not continuing the rotating spraying.

Simple and basic circuit that only requires powering resulting from the lifting or lower of the system, which can be done with a single geared motor.

It is also provided to be a removable system so that it can be arranged in multiple vats, be dispensed with, or to use a single element for several vats in a sequential manner.

The entire assembly is the fundamental objective of the invention, along with its simple regulation, without having to provide auxiliary elements.

It achieves operating uniformity, thereby conferring to the invention, as a highly effective and high-performance element, with maximum simplicity and the ability to be adapted to any vat, as it is an external element that only requires the possibility of upper access to the vat.

The invention relates to a novel apparatus and method for improving wines produced by the controlled fermentation of fruits broken up in fermentation vats by the pumping over and punching down of the must with respect to the cap and a better, greater, and controlled recirculation of the mix, improving the extraction or transfer of the active ingredients of greatest interest for fermentation of the fruits.

For this purpose, the inventor has proposed the mentioned punch down apparatus, consisting of the combination of the making use of the pump-over device for pumping over of the must and the mass of the punch down apparatus itself increased with the mass of a pointed pendulum which opens up a passage for the system, and the method of use.

The proposed punch down apparatus consists of a rotating sprayer that is axially movable along its shaft, also provided with a pointed pendulum as a breaking element, which starts breaking up the cap.

For that purpose, as a preferred but non-limiting description, it is configured in a first element as a telescopic rod which receives the must from the lower part of the vat through a pump-over device for fluids and viscous substances. This telescopic rod is introduced through the upper mouth of the vat or tank and is preferably centered with respect to same.

In summary, for re-mixing the content of a vat the punch down apparatus comprises a vertical telescopic rod, terminated in a lower mass (generally a pointed pendulum). The telescopic rod is connected to a liquid pump-over device in the lower part of the vat by means of a hose. The telescopic rod has perforations communicating with the interior of a hollow rotating station coupled in an airtight manner and with freedom of rotation to the telescopic rod above the lower mass. This rotating station has pressurized outlet nozzles for the pumped fluid. This entire assembly is connected to a system for forward and backward movement in the vertical direction.

The outlet nozzles are preferably connected to respective hollow, radial, pivoting arms (having a horizontal axis) on the rotating station. These arms have boreholes oriented downwards and inclined such that the propelling of fluid through the boreholes causes the assembly of the rotating station to rotate by a jet propulsion mechanism. The cap of vats of larger diameters can thereby be affected.

The telescopic rod is preferably formed by two parts: an upper main rod and a central rod connected to the rotating station and to the lower mass. The central rod is located inside the main rod in the stowed position.

Thus, the system for forward and backward movement can be formed by a support structure of an upper combo drive roller, connected to a geared motor. The combo drive roller comprises a notch corresponding to the outer section of the main rod and with an opposing or antagonistic pulley to ensure contact with the main rod. It also comprises circumferential grooves for taking up one or more cables connected to the central rod and passing through cable stops attached to the main rod.

The perimeter developments of the grooves and of the notch allow the forward or backward movement of the main rod coordinated with the forward or backward movement of the cable. The central rod can thus be extracted from the main rod at the same linear speed, or with a constant ratio between both speeds.

The use of a pendulum as lower mass allows the resistance of the cap not to affect the movement of the telescopic rod. Thus, the position of the latter does not vary and the telescopic rod continues to be lowered while the cap is still not broken up.

For the purpose of controlling the depth to which the system is to be introduced in the vat, respective end and safety stops are arranged, appropriately placed on the cables, appropriately placed in the telescopic rod, or by means of controlling the rotation of the combo drive roller, or other system. Said stops must be adjustable or detachable as a determining factor for the various system adjustments. If the stops are suitably adjusted, it is even possible to prevent the lower mass from piercing the cap, with the operations being reduced to pumping over of the must.

The operation of the most complete version starts with placing the support structure in the upper mouth of the vat, such that the rotating station is inside the vat, with the system being complete, the aperture and fixing of the radial arms being at 90°. The pump-over device for fluids is started up; the hoses are filled; the telescopic rod is also started up; the must exits through the perforations of the central rod and enters the rotating station. It passes through the outlet nozzles to the radial arms and through the perforations thereof, it exits under pressure against the cap and the vat; in an inclined outlet, the jets exert a tangential force causing the rotating station to rotate. In this manner, the spraying of the cap by pumping over and with automatic rotation commences, sweeping across the entire cap.

The invention further improves the system by activating the element for forward or backward movement, causing the lower mass, preferably a pointed pendulum, to descend, penetrate, and break up the cap. Once the radial arms reach the cap, the latter stops rotation by friction, and the arms perform an effect of cutting up the cap. Once the radial arms have made it past the cap, they start rotating again and move around and re-mix the fluids and skins. The system for forward movement is halted when it reaches the lower end stop provided.

By reversing the geared motor, the combo drive roller tenses the cables pulling on the assembly, and the radial arms once again encounter the cap, generally in a random manner, cutting it up again in a similar manner, but in reverse with respect to before, and when it exits the cap, the system sprays the must again over the cap, and when reaching the upper end stop the forward movement of the system stops.

Said operation can be repeated as many times considered necessary either manually or automatically, or by means of the different automatons existing on the market, ending with stopping the pump-over device and the exit from the vat so that it can be transferred to another vat, be stored, or be cleaned.

To extract the system from the vat, the arms must be folded and the assembly extracted through the mouth of the vat.

These factors enhance and improve each and every one of the features of the systems developed up until now, providing maximum value exceeding what is currently known.

Other variants will be described in the final section of this description.

### DESCRIPTION OF THE DRAWINGS

For this purpose and to complement the description being made, to aid in better understanding the features of the invention a set of drawings is attached as an integral part of said description, wherein the following has been depicted with an illustrative and non-limiting character:

FIG. 1 shows a general view of the assembly mounted in a vat.

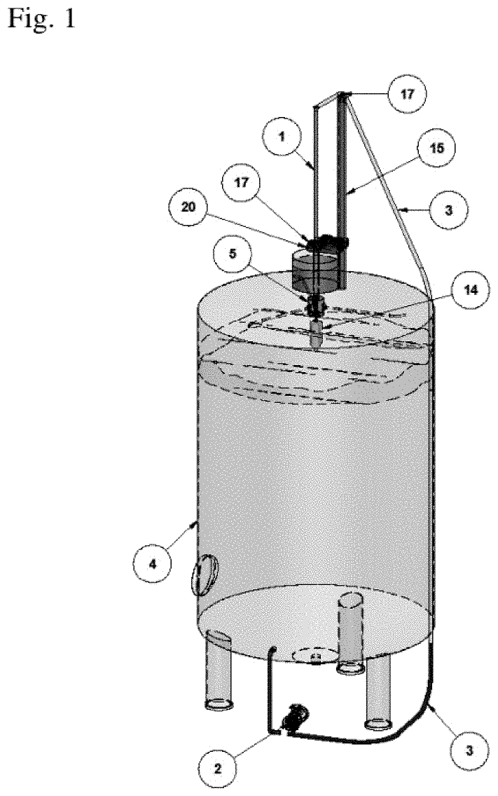

FIG. 2 shows a detail view of the punch down apparatus and automatic rotating sprayer.

FIG. 3 shows a detail view of the rotating station and the coupling of the radial arms.

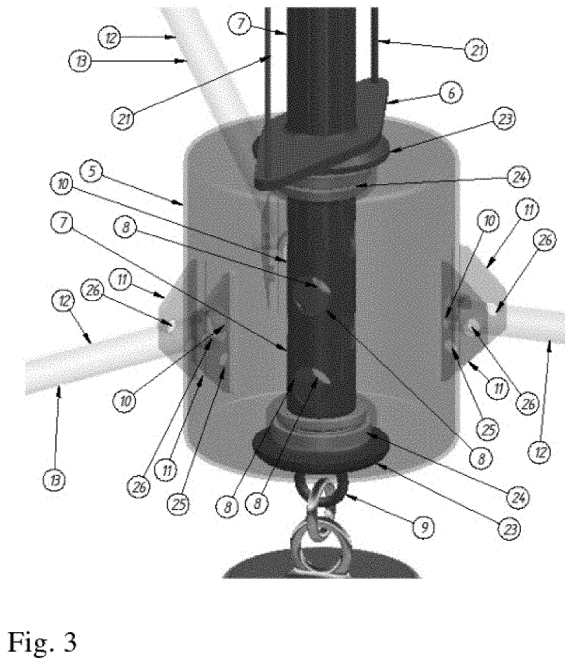

FIG. 4 shows a detail view of the element for linear forward and backward movement of the system.

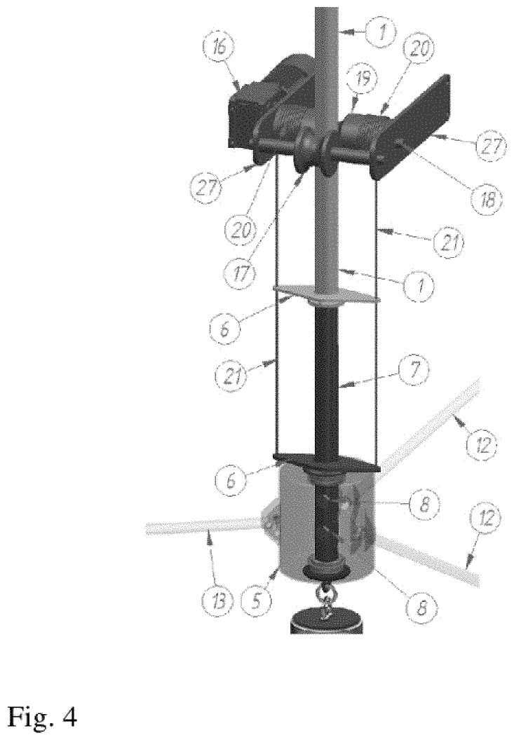

FIG. 5 shows a view of the lowering of the radial arms for extracting or introducing the apparatus through the mouth of the vats.

In said drawings, various parts or components are referenced as follows:
1.—Main rod.
2.—Pump-over device (for fluids and viscous substances).
3.—Hose.
4.—Vat.
5.—Rotating station.
6.—Cable stops.
7.—Central rod.
8.—Perforations (in the central rod).
9.—Ring.
10.—Outlet nozzles.
11.—Couplings.
12.—Arms.
13.—Boreholes (in arms).
14.—Lower mass.
15.—Support structure.
16.—Element for forward or backward movement.
17.—Pulley.
18.—Combo drive roller.
19.—Notch.
20.—Grooves.
21.—Cables.
22.—End stops.
23.—Stops (rotating station).
24.—Rolling and airtightness systems.
25.—Locking elements.
26.—Pivoting articulation.
27.—Element for forward or backward movement.

### PREFERRED EMBODIMENT OF THE INVENTION

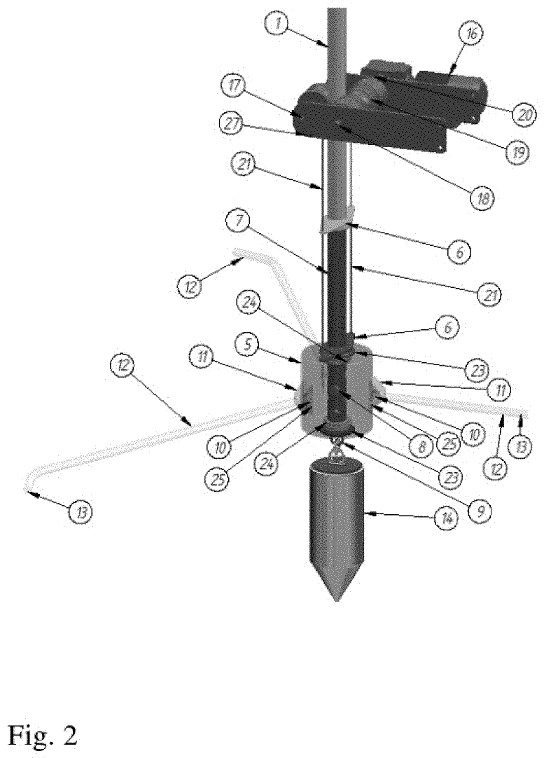

In view of the mentioned figures, it can be seen how the proposed invention, a punch down apparatus-automatic rotating sprayer, (FIG. 2), is preferably made up of a main rod or tube (1) receiving from the pump-over device (2) for fluids and viscous substances the must from the lower part of the vat (4) through a conduit or hose (3), which is generally flexible in some or all sections thereof.

The main rod (1) has coupling elements for coupling the hose (3) at one end. At the other end, the main rod (1) has coupling elements for coupling a central rod (7) supporting at its free end a rotating station (5) with freedom of rotation. The central rod (7) is vertically mobile, such that it can be extended from inside the main rod (1) outwards and downwards, or be drawn into the interior thereof, forming an airtight telescopic rod (1, 7). The main rod (1) may also comprise respective removable end stops (22) which, upon contact, detains or reverses the forward or backward movement direction of the system.

The central rod (7) has perforations (8) in the portion farthest away from the main rod (1), inside the rotating station (5), such that the must or fluid can exit the interior of same into the rotating station (5). The connection between the central rod (7) and the rotating station (5) is performed by rolling and airtightness systems (24) so that must or fluid does not leak out. The perforations (8) of the central rod (7) allow the fluid to exit under pressure or pumped must and the filling and pressurizing of the rotating station (5).

The main rod (1) has one or more integral cable stops (6) for one or more cables (21) which allow hoisting the assembly of the main rod and the central rod (7) from an upper combo drive roller (18).

The central rod (7) is limited to its axial movements from the main rod (1) by respective stops (23). The lower stop (23) is terminated in a coupling in the form of a ring (9) or anchoring.

The rotating station (5) has couplings (11) on its periphery which support several outlet nozzles (10), three in the figures. These couplings (11) have hollow, radial arms (12), the number of same being equal to the outlet nozzles (10), and anchored by pivoting articulations (26) to the couplings (11). The arms (12) have boreholes (13) that are oriented downwards and inclined, such that the propulsion of the must or fluid jets causes the assembly of the rotating station (5) to rotate. It is also possible to place boreholes (13) in other positions, for example at the ends of the arms (12), oriented towards the inner wall of the vat (4) for cleaning.

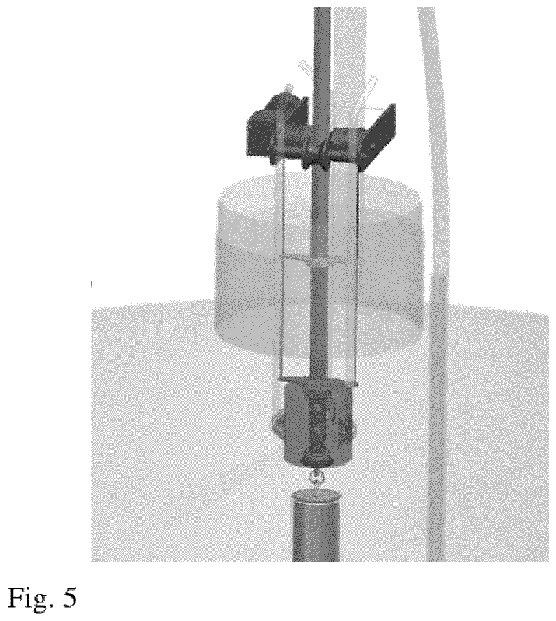

The arms (12) can be locked in the couplings (11) of the rotating station (5) by locking elements (25) in the deployed position (FIG. 2) and therefore rotate with same. They can also be placed in the position of FIG. 5, approximately parallel to the central rod (7), to facilitate storage and the entry in and exit from the vat (4).

The ring (9) allows receiving a lower mass (14), in this case a pendulum terminated in a pointed manner, the dropping of which can break up the cap. This lower mass (14) can be attached to the central rod (7) by another method. For example, it may be integral with the central rod (7) below the rotating station (5).

The invention comprises a support structure (15) as a bearing element supporting a pulley (17) to hold the hose (3) and the system (27) for forward or backward movement of the central rod (7).

The element (27) for forward or backward movement of the system in the axial or vertical direction is equipped with a geared motor (16) or the like which operates the combo drive roller (18). The combo drive roller (18) can rotate in either direction depending on the operation of the geared motor (16). In addition to taking up the cable (21), this combo drive roller (18) drives the main rod (1) in an upward movement. To that end, a freely rotating roller or pulley (17) ensures that the main rod (1) is clamped against the combo drive roller (18) and can be moved by same.

The central part of the drive roller (18) is configured with a notch (19) which defines the “negative” or outer section of the main rod (1) such that it generates a surface which drives the main rod (1) upon rotation. It also has respective circumferential grooves (20) on both sides for winding the cables (21). The perimeter development of the grooves (20) and of the notch (19) allows the forward or backward movement of the main rod (1) coordinated with the forward or backward movement of the cable (21).

The associated cables (21) go through cable stops (6) arranged in the main rod or tube (1), to terminate or end up being rigidly fixed to the cable stops (6) arranged and integral with the central rod (7), which provides a bearing for the entire system of the pumping over, automatically rotating, punch down apparatus.

The punch down apparatus can be supported by a gantry or any other type of crane for being placed above the different vats (4) of a winery.

1. A punch down apparatus for re-mixing a content of a vat (4) comprising:
a vertical telescopic rod (1, 7) terminated in a lower mass (14), connected to a pump-over device (2) in the lower part of the vat by a hose (3) and having perforations (8) communicating with the interior of a hollow rotating station (5) coupled in an airtight manner and with freedom of rotation to the telescopic rod (1, 7) above the lower mass (14), the rotating station (5) having pressurized outlet nozzles (10) for the pumped fluid a system (27) for forward and backward movement of the assembly in the vertical direction.

2. The punch down apparatus according to claim 1, wherein the outlet nozzles (10) are connected with respective hollow, radial, pivoting arms (12) which have boreholes (13) oriented downwards and inclined such that the propelling of fluid through the boreholes (13) causes the assembly of the rotating station (5) to rotate.

3. The punch down apparatus according to claim 1, wherein the telescopic rod (1, 7) is formed by an upper main rod (1) and a central rod (7) connected to the rotating station (5) and to the lower mass (14).

4. The punch down apparatus according to claim 3, wherein the system (27) for forward and backward movement is formed by a support structure (15) of an upper combo drive roller (18), connected to a geared motor (16), and comprising:
a notch (19) corresponding to the outer section of the main rod (1) and with an opposing pulley (17) to ensure contact with the main rod (1) and
circumferential grooves (20) for taking up one or more cables (21) connected to the central rod (7) and passing through cable stops (6) attached to the main rod (1);
such that the perimeter developments of the grooves (20) and of the notch (19) allow the forward or backward movement of the main rod (1) coordinated with the forward or backward movement of the cable (21).

5. The punch down apparatus according to claim 1, wherein the lower mass (14) is a pendulum.

6. The punch down apparatus according to claim 1, wherein the lower mass (14) is pointed.
